(19) United States
(12) Patent Application Publication
(10) Pub. No.: US 2014/0012180 A1
(43) Pub. Date: Jan. 9, 2014

(54) PERITONEAL DRAIN AND INFUSION

(71) Applicant: Nidus Medical, LLC, Atherton, CA (US)

(72) Inventors: Howard LEVIN, Teaneck, NJ (US); Mark GELFAND, New York, NY (US)

(73) Assignee: Nidus Medical, LLC, Atherton, CA (US)

(21) Appl. No.: 13/858,403

(22) Filed: Apr. 8, 2013

Related U.S. Application Data
(60) Provisional application No. 61/640,935, filed on May 1, 2012.
(60) Provisional application No. 61/640,965, filed on May 1, 2012.

(51) Int. Cl. A61M 27/00

(52) U.S. Cl. CPC A61M 27/002
USPC 604/9

(57) ABSTRACT

A method to remove ascites from a mammalian body including: draining ascites from the peritoneal cavity to the bladder of the mammalian body, and evacuating the drained ascites out of the bladder. The method may be practiced with a valved fistula implanted into the bladder to form a passage between the peritoneal cavity and the bladder.

### CROSS-REFERENCE TO RELATED APPLICATIONS

This application claims the benefit of priority to U.S. Prov. 61/640,935 filed May 1, 2012 and 61/640,965 filed May 1, 2012, each of which is incorporated herein by reference in its entirety for any purpose.

### BACKGROUND OF THE INVENTION

This invention relates to methods for treating ascites such as by implanting an artificial fistula device to drain ascites fluid.

Ascites is the accumulation of ascetic fluid, which is usually a serous fluid that is a pale yellow or a clear fluid. Ascetic fluid accumulates in the abdominal (peritoneal) cavity. Ascitic fluid may result from liver disease, cancer, congestive heart failure and kidney failure.

Ascites is traditionally classified as transudative or exudative. This division of ascites is based on the amount of protein in the ascetic fluid. The Serum Ascites Albumin Gradient (SAAG) is another system developed to classify ascites based on the amount of albumin in the ascitic fluid as compared to the serum albumin (albumin measured in the blood). Ascites related to portal hypertension (cirrhosis, congestive heart failure, Budd-Chiari) typically generates ascites fluid having a SAGG of greater than 1.1. Ascites caused by other reasons (malignant, pancreatitis) has an SAGG that is lower than 1.1.

There may be no symptoms associated with ascites especially if it is mild. Mild ascites is usually less than about 100 milliliters (ml) to 400 ml of accumulation of ascites fluid in an adult. Abdominal pain, discomfort, and bloating are frequently seen as the amount of ascites increases beyond 100 to 400 ml of accumulated fluid in an adult. As more fluid accumulates, increased abdominal girth and size are commonly seen. A cosmetically disfiguring large belly, due to ascites, is a common concern of some patients.

Shortness of breath can occur as increasing volumes of ascites increase pressure on the diaphragm. The migration of she ascites fluid across the diaphragm can cause pleural effusions, which includes fluid accumulating around the lungs.

Patients suffering from severe ascites are conventionally treated in a hospital, doctor's office or other medical facility. These patents undergo paracentesis which involves extracting ascites fluid from the abdomen. During the procedure, patients are asked to lie down and expose their abdomen. After cleaning the side of the abdomen with an antiseptic solution, a physician numbs a small area of skin and inserts a fairly large-bore needle (along with a plastic sheath) having a diameter of 2 to 5 cm into the abdomen and extends the needle and sheath to the peritoneal cavity to reach the ascetic fluid. The needle is removed, leaving the plastic sheath through which the ascites fluid will drain. The fluid can be drained by gravity or by connection to a vacuum bottle. Up to 11 litres (L) of fluid may be drained during the procedure. If fluid drainage is more than 5 litres, patients may receive intravenous serum albumin (25% albumin, 8 g (grams)/L) to prevent hypotension (low blood pressure)

The procedure to drain the ascites fluid is generally not painful and patients require no sedation. As long as they are not too dizzy and maintain their blood pressure after the procedure, patients go home promptly after the procedure.

It is also know how to drain ascites into the venous blood circulation. A LeVeen peritoneovenous shunt includes a surgically implanted subcutaneous plastic tube to provide continuous shunting of ascites fluid from the peritoneal cavity to the jugular vein. The required surgery to implant the LeVeen peritoneovenous shut is risky and difficult. There is also a risk that the shunt will malfunction due to clogging of the plastic tube and high venous pressure. The exposure of the shunt to blood raises a risk of systemic infections. The LeVeen shunt is typically used in refractory patients who have not responds well to serial therapeutic paracentesis and are not candidates for liver transplant.

Peritoneal Dialysis Catheters are known for the treatment of renal failure. Catheter malfunction is a common complication of Peritoneal Dialysis, occurring in 15-30% of patients, and is a common cause of catheter loss. The proper functioning of a peritoneal catheter with unrestricted flow of dialysate solution can be compromised by catheter malposition or kinking, constipation, a fibrin clot, omental wrapping, or obstruction secondary to intraperitoneal adhesions.

U.S. Pat. No. 7,335,179 to Burnett (reported to be assigned to Novashut AG and related patents) discloses a transvesicular drainage device, designed to drain excess fluid from the peritoneal cavity and other locations in the human body to the bladder. The device consists of a long tube with a debris filter placed in the peritonea cavity and the unidirectional vale. The device is surgically placed by a surgeon through an opening made in the abdominal cavity.

### BRIEF DESCRIPTION OF THE INVENTION

A novel way to relieve ascites in a patient has been invented. At the bottom of the peritoneal cavity the peritoneum (membrane) abuts the urinary bladder. The inventors take advantage of this anatomic phenomenon to eliminate the need for connecting tubes and complex surgery associated with certain approaches, e.g., the LeVeen short.

The inventors conceived of and developed a minimally invasively artificial fistula to be surgically or otherwise minimally invasively inserted and implanted between the peritoneal cavity and bladder. Ascites drains from the peritoned cavity, through the fistula and into the bladder. The ascites can be evacuated from the bladder during urination. The fistula is equipped with a one way valve to prevent backflow of fluid from the bladder into the peritoneal cavity.

The fistula allows ascites to flow from the peritoneal cavity due to the force of gravity. The patient may assist or control evacuation of ascites from The peritoneal cavity by tensing the abdominal muscles and increasing intra-abdominal pressure with or without simultaneous urination. The muscles of the patient form a “muscle pump” which the patient controls to push ascites through the fistula. The increased pressure forces ascites through the fistula and into the bladder. The flow of ascites is controlled by a simple and reliable muscle pump that is activated by the patient to opens the valve in the fistula and propel the ascites into the bladder. Mechanical pumps may be unnecessary to move the ascites from the peritoneal cavity to the bladder.

The bladder acts as a temporary storage reservoir for urine generated by the kidney. The bladder walls contain a muscle called the detrusor, which contracts to generate pressure in the bladder to expel urine.

The bladder wall also contains stretch receptors, which send signals about the distension of the bladder to the spinal cord. The interval of time between episodes of urination depends on the available volume of the reservoir in the bladder. In normal adults, the capacity of the bladder is at least 500-700 cc (cubic centimeters). As the amount of fluid in the bladder approaches the capacity of the bladder, the spinal cord reacts so the signals from the stretch receptors by activating the detrusor muscle. A person with an intact nervous system will be aware of both the distension and the pressure produced by the muscle contractions. If it is inconvenient to urinate, the person can voluntarily contract the sphincter muscle to prevent urination until it is convenient to do so. Urination is permitted to occur by relaxing the sphincter. A mean bladder wall thickness is generally 3.0 to 4.0 mm (millimeters) in healthy women and men.

The abdominal cavity is located below the chest cavity, separated from it by the diaphragm. The abdominal cavity the space bounded by the vertebrae, abdominal muscles, diaphragm and pelvic floor) is distinct from the intraperitoneal space (located within the abdominal cavity, but wrapped in peritoneum). For example, a kidney is inside the abdominal cavity, but is retroperitoneal.

The peritoned cavity is the space between the layers of the peritoneum. The outer layer of the peritoneum, called the parietal peritoneum, is attached to the abdominal wall. The inner layer, the visceral peritoneum, is wrapped around the internal organs that are located inside the intraperitoneal cavity. The space between these two layers is technically outside of the peritoneal sac. The peritoneal cavity generally refers to either or both the space between the layers of the peritoneum and the peritoneal sac.

The peritoneal cavity is filled with a small amount (about 50 ml) of slippery serous fluid that allows the two layers of the peritoneum to slide freely over each other. The term mesentery refers to a double layer of visceral peritoneum. There are often blood vessels, nerves, and other structures between the layers of the peritoneum.

Endoscopy of the urinary bladder via the urethra is called cystoscopy. Diagnostic cystoscopy is usually carried out with local anesthesia. General anesthesia is sometimes used for operative cystoscopic procedures. Ureteroscopy is defined as upper urinary tract endoscopy performed most commonly with an endoscope passed through the urethra, bladder, and then directly into the upper urinary tract. Indications for ureteroscopy have broadened from diagnostic endoscopy to various minimally invasive therapies.

In one embodiment, the valved fistula is placed using transvesical (crossing the wall of the bladder) laproscopic surgery or using a variation of cystoscopy and ureteroscopy. The artificial fistula may be placed using laparoscopic surgery, or interventional radiology tools that pass through a small incision or puncture in the skin of the patient. Various laparoscopic tools are used to puncture the pathway for the valved fistula and to secure the device in the desired location. The surgical method disclosed herein may be envisioned as riveting together one or more layers of the peritoneum and the wall of the bladder and implanting a one way fluid channel for drainage of ascites. Ascites fluid flows from the peritoneal cavity into the bladder through the one way fluid channel when the hydrostatic pressure inside the abdominal cavity is higher than the pressure in the bladder. When the bladder pressure is more than the abdominal cavity, ascites fluid is blocked from flowing to the bladder. A relatively small pressure difference may be sufficient to open the valved fistula and allow ascites fluid to flow into the bladder.

The valved fistula need not withstand much pressure. The valved fistula may include a flexible thin walled tube. The tube may be stretchable and made of a polymer such as silicone rubber. The tube extends into the internal space of the bladder. The tube may collapse when The pressure in the bladder is the same or greater than the pressure the ascites flow through the tube. Alternatively or in addition, a valve embedded in or coupled to the tube may close when the pressure in the bladder is raised to above the ascites flow pressure.

The valved fistula may include a silicone duckbill attached to a nitinol self-expanding stent-like retainer with silicone seals. The retainer and seals secure the valved fistula to the bladder and peritoneal membrane around the valve. The retainer and seals also occlude the opening made in the layers of the peritoneum and the wall of the bladder.

A duckbill valve may be manufactured from rubber or synthetic elastomer. The duckbill valve may be shaped in side view somewhat like the beak of a duck. The shape of the duckbill valve may differ from the beak of a duck in alternative embodiments.

The duckbill valve may have a flexible tubular end which is stretched to fit over the outlet of a fluid supply, conforming itself to the shape of the valve inlet, usually annular or round. The other end, the duckbill, retains its natural flattened shape. When a fluid is pumped through the supply line and therefore the duckbill, the flattened end opens to permit the pressurized fluid to pass. When pressure is removed, however, the duckbill end returns to its flattened shape, preventing backflow.

A duckbill check valve is an example of a suitable valve for the valved fistula. Duckbill check valves allow free fluid flow when opened by a positive differential pressure across the fluid path through the valve. The duckbill check valve is closed by a negative differential pressure so that backflow through the valve is prevented. The tube need not have internal parts that promote clotting with solids. The passage of fluids and solids through the fistula with a duckbill check valve may be facilitated and controlled by the patient voluntarily engaging abdominal muscles to raise the intra-abdominal pressure or by application of external pressure to the abdomen. To further prevent back-flush of urine through the valved fistula the passage through the fistula may include a plurality of valves in arranged series along the length of the passage.

Duckbill check valves may be designed to operate in response to a small pressure change of a few millimeters of mercury depending on valve size, geometry and compound characteristics. Duckbill check valves are also designed to function at specific opening and closing pressure ranges, depending on specifications. Sizes for duckbill valves can range for example from 2.5 mm to 10.0 mm in diameter.

Compared to catheters and other prior art, an implantable fistula device to drain ascites into the bladder of the patient may be resistant to clotting by solids, not require complex or risk prone surgery, and not require implantable pumps and battery power systems for such pumps.

An implantable device, e.g., fistula, to drain ascites into the bladder of the patient should be relatively indifferent to clotting by solids. Such an implantable device should not require complex or risk prone surgery and preferably avoid implantable pumps and the battery power systems to power pumps. The complications of a peritoneal catheter are addressed with the fistula disclosed herein by minimizing the part of the fistula residing in the peritoneal cavity. Anastomisis of the valve to the intraperitoneal surface can be performed by stapling or suturing that minimizes the irritation and adhesions. The absence of the intraperitoneal tube eliminates kinking. Relatively large bore short passage is intended to minimize constipation.

### BRIEF DESCRIPTION OF THE DRAWINGS

FIG. 1 is an illustration of a valved fistula implanted in the urinary bladder to form a passage between the peritoneal cavity and bladder.

FIG. 2 is an illustration of a surgical method to implant the valved fistula using a laparoscopic instrument into the bladder cavity through the urethra.

FIG. 3 is an illustration of a distal end of a laparoscopic instrument extending through the wall of a bladder and into the peritoneal cavity.

FIGS. 4A and 4B illustrate embodiments of an implanted valved fistula.

FIG. 5 is an illustration of an implanted valved fistula with tubes having internal one way valves.

FIG. 6 is an illustration showing a patient having a subcutaneous infusion port that drains fluid from the peritoneal cavity patient to the bladder after an infusion of osmotic fluid to the peritoneal cavity.

FIGS. 7 and 8 are illustrations of a male and female in which a laparoscopic instrument is being used to insert a valved fistula through a urinary tract.

FIGS. 9 to 13 are illustrations sequentially showing the implantation of a fistula into the walls of the bladder and peritoneal cavity.

FIGS. 14 to 18 are illustrations of connecting valves to the implanted fistula.

FIG. 19 is an illustration of the components for the fistula and tubular valve.

FIGS. 20 to 27 are illustrations sequentially showing the implantation of the fistula into the walls of the bladder and peritoneal cavity.

### DETAILED DESCRIPTION OF THE INVENTION

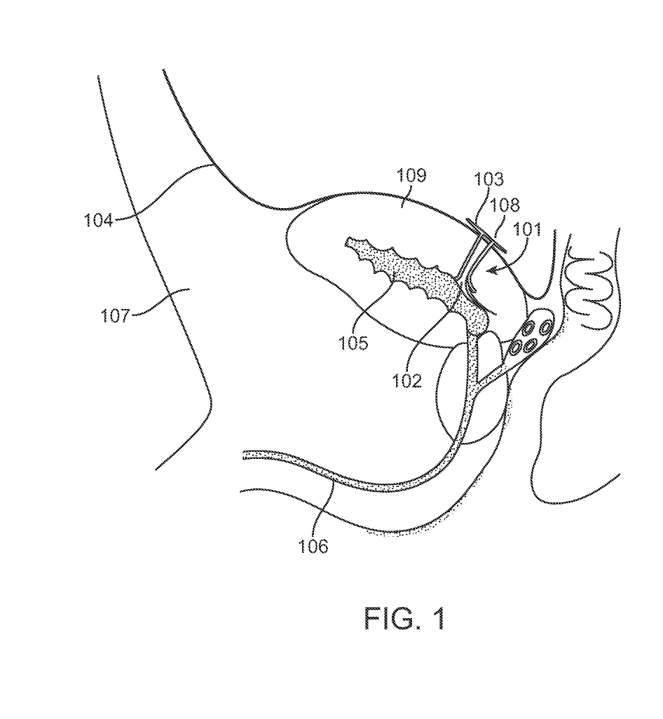

FIG. 1 shows one embodiment of an implanted valved fistula 101 forming a substantially unidirectional fluid flow path between the peritoneal cavity 108 and the urinary bladder 105. Ascites that collects in the peritoneal cavity 108 is conveyed to the bladder through the fistula. A valve in the fistula may be activated by a pressure differential between the peritoneal cavity and the bladder. The pressure differential may be created by the force of gravity alone or a combination of gravity and the contraction of muscles in the abdominal wall 107 of the patient.

To discharge the ascites from the bladder, the patient may contract the bladder 105 and evacuate the ascites mixed with urine though the urethra 106. The urethra is a tube connecting the bladder to the outside of the body. The external urethral sphincter is a striated muscle that allows voluntary control over urination as well as the evacuation of ascites from the bladder.

The valved fistula 101 includes a retention element having annular discs 101 and a tubular structure 102 that forms a pathway through the peritoneal membrane 104 and the muscle wall 109 of the bladder 105. The tubular structure 102 provides a fluid passage for ascites to flow from the peritoneal cavity to the bladder. The retention element 103 is secured to and retained by the peritoneal membrane and muscle wall of the bladder.

A tubular element 102 is integral with or attached to the retention element 103. The tubular element forms a valve by being a collapsible tube or a duckbill. A function of the valve is to prevent passage of urine from bladder to peritoneal cavity when the bladder contracts during urination. The valve allows ascites to flow into the bladder. The valve may be configured to allow ascites to flow from the peritoneal cavity to the bladder without any conscious action by the patient. Alternatively, the valve may be configured to allow flow of ascites from the peritoneal cavity to the bladder when, e.g., only when, the patient activates the valve, such as by contraction of the muscles in the abdomen or stomach to increase the pressure in the peritoneal cavity.

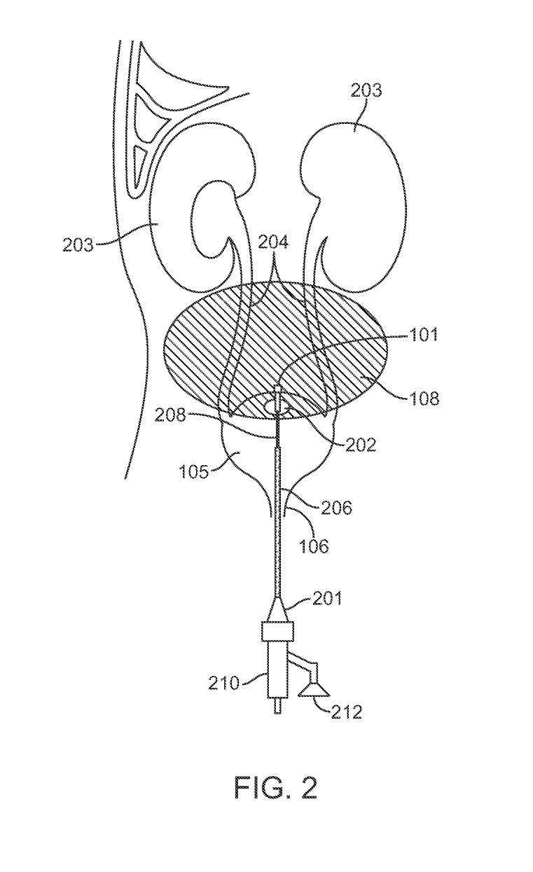

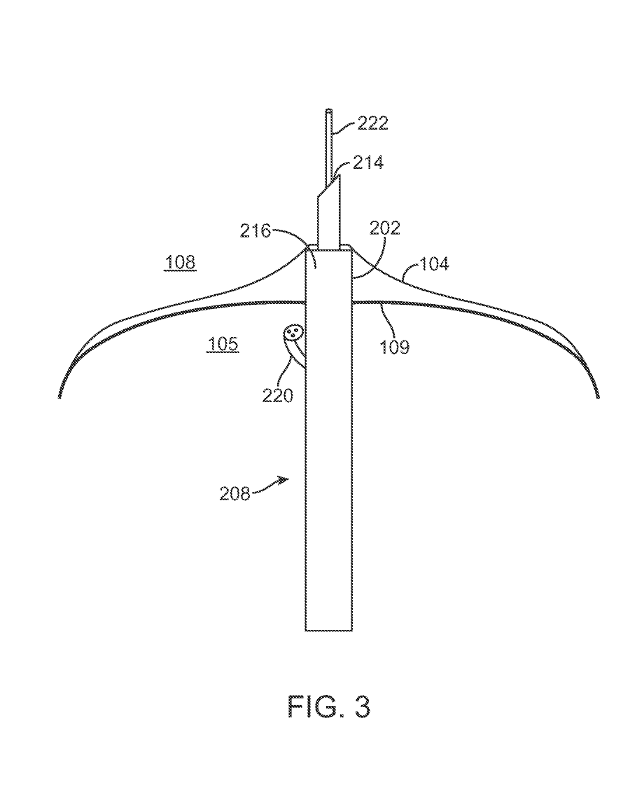

FIGS. 2 and 3 illustrate a surgical method of installing the fistula 101 (shown schematically) using a laparoscopic instrument 201 introduced into the bladder 105 through the urethra 106. The bladder 105 is connected to the kidneys 203 via the right and left ureters 204. The kidneys 203 are in the abdomen outside of the peritoneal cavity.

The laparoscopic instrument 201 may include a multi-lumen catheter 206 having a distal end section 208 and a proximal end that connects to a controller device 210 for the laparoscopic instrument. The controller may include user controls for manipulating and positioning the catheter through the urethra and to the wall of the bladder, extending and retracting a guide wire and a cutting tip 214 (FIG. 3) through a passage in a hollow tube 216, e.g., a single or multi-lumen catheter tube, that forms an outer tube of the catheter, extending and retracting an inner tube or wire which moves the fistula 101 through the tube of the catheter to the bladder and implants the fistula, and observing with a display 212 images obtained by a camera lens at the distal section 208 of the catheter.

While the distal section 209 of the catheter is positioned at the muscle wall 109 of the bladder 105, the cutting tip 214 is extended from the end of the distal section and cuts an orifice 202 in the muscle wall 109 and peritoneal membrane 104. At least a portion of the catheter tube 216 is extended into the orifice 202 distal to expand the orifice to receive the fistula, which is temporarily stored in the distal section of the tube 216. The fistula will be push out from the tube and into the orifice, as the tube is retracted from the orifice. The orifice is between the peritoneal cavity 108 and the bladder 105.

The trans-urethral laparoscopic approach is not the only available implantation procedure for installation of the valved fistula. The valved fistula may be placed using laparoscopic surgery from outside of the bladder as well as by using a variation of cystoscopy and ureteroscopy. The laparoscopic instrument can be introduced through the puncture orifice in the wall of the abdomen. For example, the laparoscope can cross the anterior bladder wall and emerge through the other wall into the peritoneal cavity. A skilled surgeon can find several ways to reach the desired location where the bladder wall and the peritoneal membrane are in juxtaposition.

FIG. 3 shows schematically the distal end 208 of the laparoscopic instrument 210 (See FIG. 2). Various instruments can be used to perforate the peritoneal membrane 104 and the bladder wall 109 and puncture a hole in both. Hollow tubes, e.g., tube 216, can be used to infuse gas such as carbon dioxide into the peritoneal cavity to separate the internal organs and create a working space 218. A miniature fiber optic camera 220 may be attached to the distal section 208 to capture images of the walls of the bladder and peritoneal cavity, the implantation of the fistula, and to inspect the space and assist the procedure. A guide wire 222 may be used to exchange the parts of the fistula or the catheter, guide the parts of the fistula into the orifice, and assist the surgeon in creating the orifice and implanting the fistula.

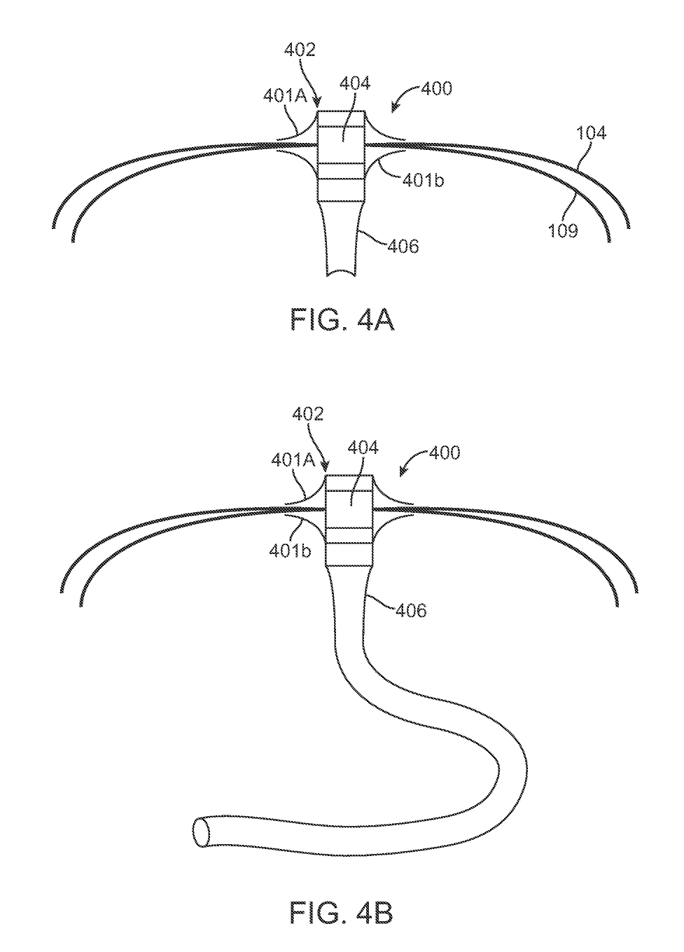

FIGS. 4A and 4B show schematically examples of embodiments of an implanted valved fistula 400, which includes a retainer mechanism 402 having retaining discs 101 and having a hollow tube 404, e.g., a flexible biocompatible polymer cylinder, which forms the passage for ascites flowing from the peritoneal cavity to the bladder. The tube 404 of the retainer mechanism 402 may be a short stent-like cylindrical structure, a metal or a plastic tube with an internal fluid passage to allow fluid to enter the device without impediment. The outer surface of the retainer tube 404 may be knurled, roughened, mesh covered, dimpled or otherwise adapted to be biocompatible, attached to the tissue surrounding the orifice, and effect closure of the orifice around the tube.

The retainer mechanism 402 may also include a retainer 401a, 401b, such as an annular disc, brim, rim, wire loops, struts, (such as nitrol struts), that extends radially outward from the tube 404. The retainer mechanism 402 is collapsed when temporarily stored in the hollow tube 216 of the laparoscopic instrument 201. When the valved fistula 101 is pushed out the distal end of tube 216, the retainer expands, such as shown in FIGS. 4A and 4B. The expansion of the retainers 401a, 401b may be akin to the opening of an umbrella.

The retainer mechanism 402 may include a lower retainer 401a that seats on the muscle wall 109 of the bladder and an upper retainer disc 401b that seats on the membrane 104 of the peritoneal cavity. The upper and lower retainers discs 401a, 401b hold the peritoneal membrane 104 and the bladder wall 109 together tightly, and thereby secure the fistula in the body of the patient. When the fistula being implanted in the orifice 202, the retainer discs 401a, 401b expands to contact the muscle wall 107 of the bladder and the peritoneum membrane 104. The opposing retainer discs 401a and 401b may grab, pierce and staple together the muscle wall and the peritoneum membrane.

The retainer mechanism 402 may be attached to a duckbill or flapper valve 404 extending into the bladder cavity, as shown in FIG. 4A. FIG. 4B shows a longer collapsible tube valve 406 that may have internal flapper valves. The longer tube 406 may be useful to provided enhanced prevention of migration of bacteria from the bladder to the peritoneal cavity.

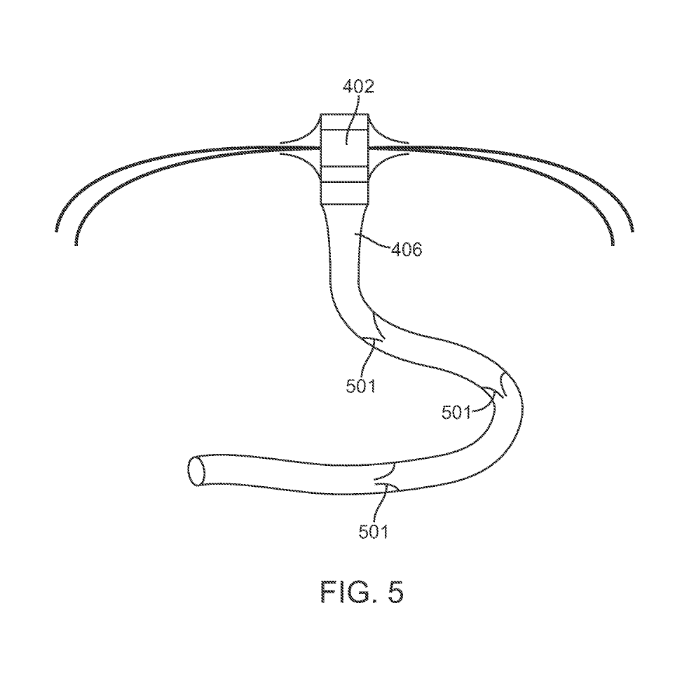

FIG. 5 shows a valved fistula having a retainer mechanism 402 and a long collapsible tube valve 406 with internal flapper valves 501. The multiple valves 501 can have an advantage of better preventing the backflow of urine into the peritoneal space and migration of bacteria from the bladder to the peritoneal cavity. The tube valve 406 may be attached to the retainer mechanism 402 after the retainer mechanism is implanted.

The retainer mechanism 402 with valved tube may be a device incorporating a one-way valve supported by a stent-like self-expanding retainer that secures the valve in place during all physiological conditions, including bladder contraction. The retainer discs 401a, 401b maybe a self-expanding tubular mesh structure formed from Nitinol super-elastic alloy tubing and processed to its final expanded dimensions. The retainer discs 401a, 401b may be covered with silicone to create a seal between the retainer mechanism 402 and the live tissue of the muscle wall 109 and peritoneal membrane 104. The silicone membrane may be a coating formed integrally with the retainer, the passage and the valves which are the structures exposed to live tissue and the flow ascites and other body fluids. Other polymer a biologic materials can be used to coat the retainer mechanism. The valves in the valved fistula may be made of bovine or porcine peritoneum, or for example a bovine jugular venous valve. Bovines have native valves in their veins.

The propulsion of material and ascites from the peritoneal cavity to the bladder may be assisted by the patient trained so perform voluntary maneuver similar to the Valsalva Maneuver. To perform this maneuver, the patient inhales, hold his breath, and bears down while tightening the chest and abdominal muscles as is they were straining while having a bowel movement. While bearing down, the patient suddenly releases and breathes out. During this maneuver, the contraction of the abdominal muscle will evacuate solids that can clog the valve of the fistula and overcome any resistance of the valve to opening.

It may be desirable to design the valve in the valved fistula so that it requires 5-10 or more mmHg of intra-abdominal pressure to open. This will ensure that valve is only opened during the voluntary evacuation maneuver and there is no leakage of fluid retrograde from the bladder. It is anticipated that the valved fistula may include several valves in series to further prevent such back flush.

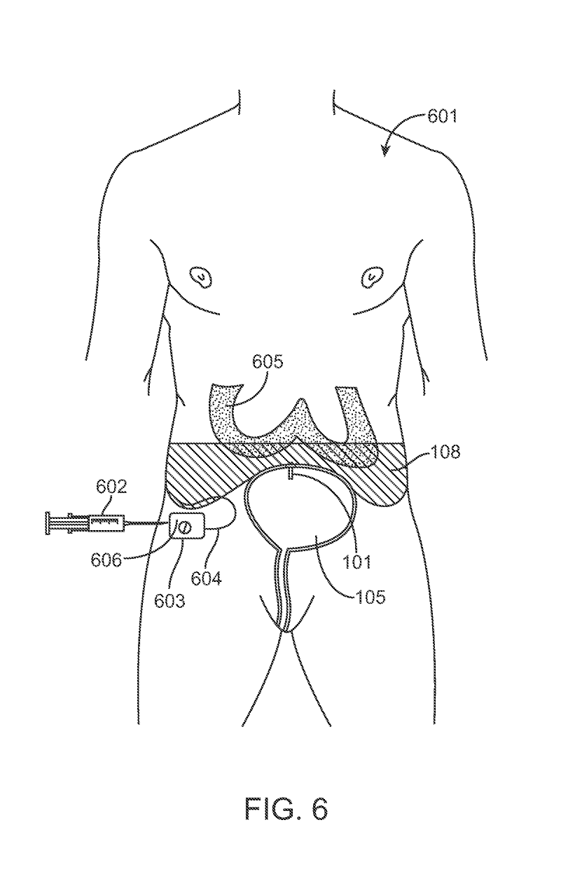

FIG. 6 illustrates a human (or other mammalian) patient 601 having a subcutaneous infusion port 603 through which fluid is infused to the patient, such as into the abdomen or directly to the peritoneal cavity. The fluid infused through the port 603 may be an osmotic fluid. An influx of body fluid into the peritoneal cavity is temporarily induced by the infusion of osmotic fluid from the infusion port 602 and into the peritoneal cavity. A valved fistula 101 removes fluid from the patient by promoting a fluid shift to the peritoneal cavity and providing drainage of the fluid into the bladder 105. The port 602 and the fistula 101 may be integrated in a single implantable device.

The subcutaneous infusion port 603 may be connected to the peritoneal cavity 108 via an infusion catheter 604. Osmotic material dissolved in water forms the osmotic fluid that is moved by a syringe 602 through the port 603 and into the peritoneal cavity. Body fluid is attracted by the infused osmotic material and accumulates in the peritoneal cavity 108 where it may at least partially surround the bowels 605. The body fluid accumulated in the peritoneal cavity is drained to the bladder 105 through the valved fistula 101 and subsequently evacuated from the bladder by urination.

Infusion of fluids with high osmolarity for therapeutic purposes into the human peritoneum is known. Peritoneal Dialysis (PD) requires large volumes of fluid exchange. Tenckhoff Chronic Peritoneal Dialysis Catheters are typically used for chronic access to the peritoneal cavity.

The embodiment illustrated in FIG. 6 shows, for the purpose of an example, an implanted subcutaneous infusion port 603 based on the design of a commercially available Port-A-Cath™ device offered by Deltec, Inc. of St. Paul, Minn., that may be connected to an already implanted infusion catheter 604 for repeat infusions of fluid into the cavity. Implanted ports 603 are generally less vulnerable to infection and more comfortable to the patient than catheters extending externally from the patient.

If less frequent skin punctures are desired, the port 603 may be an implantable pump with a reservoir of the osmotic fluid. Such pumps are frequently used to infuse pain controlling medication in chronically ill with refractory pain. The osmotic solution can be periodically infused by physician into a covering of an artificial septum 606 or other internal reservoir of the pump and subsequently delivered from the septum 606 into the peritoneal cavity slowly and gradually, such as in a continuous drip flow or infused at scheduled intervals, such as the infusion of an implanted pump with a computer controller may provide the gradual or scheduled delivery of the osmotic fluid to the peritoneal cavity to provide continuous ambulatory peritoneal dialysis (CAPD).

The amount of osmotic fluid to be infused from the infusion port 603 may be small as compared to peritoneal dialysis. The infusion catheter 604 may have a relatively small lumen, such as 8 French or smaller, to infuse the fluid from the port to the peritoneal cavity. The infusion port 603 may have a silicon puncture able septa to periodically, such as once a day or once a week, receive a small diameter needle of the syringe 602.

A device conventionally used as a vascular access graft or similar device may be as used in or as the infusion port 603. The graft may be made of a material that closes after being punctured by a needle by mechanical strength rather than by blood clotting. This graft will allow a much larger area for puncturing resulting in increased lifetime of the device.

Common to all these described embodiments of the infusion port is that the risk of infection is reduced as compared to a Tenckhoff type catheter that crosses the skin. Comfort of the patient is increased and the risk of undesired puncture of a bowel or a blood vessel is decreased compared to a transcutaneous needle puncture that is commonly used in paracentesis.

It is appreciated that the peritoneal dialysate fluid is a growth medium for bacteria and bacteria easily form biofilm on the surface of the catheter(s). Bacteria entering the catheter and/or the peritoneal cavity can proliferate fast and cause peritonitis, a life threatening complication. The principal portal of entry for bacteria and other organisms is thought to be the external surface and lumen of the catheter. The commercial production of peritoneal dialysate and other sterile infusion fluids under stringent quality control criteria makes this fluid itself an unlikely source of bacteria. The commonest cause of infection is touch contamination during connecting or disconnecting a fresh dialysate bag.

In the proposed embodiment(s), injections of an osmotically active substance may be relatively infrequent and performed by competent medical personnel in a medical clinic or a doctor's office, to reduce the risk of touch contamination that is common in home dialysis. Alternatively, osmotic material may be infused via the port into an implanted reservoir associated with the infusion port 602. The stored osmotic material is released at different times into the peritoneal cavity 108 as needed, minimizing the number of port accesses, and lowering the risk of infection.

In addition to the removal of ascites, the disclosed embodiments of the implanted valved fistula may facilitate peritoneal removal of fluid from body cavities and spaces other than peritoneum itself, such as the legs, lungs and other large extremities or body organs. To make this treatment available to the large number of patients in need such as, for example, CHF patients with diuretic resistance and fluid overload and liver disease patients that have not developed large volume ascites. By infusing osmotic solution into the peritoneal cavity, fluid is drawn from other spaces into the peritoneal cavity. The valved fistula 101 connecting the peritoneal cavity to the bladder allows the patient to remove said fluid without assistance from physician using the natural pathway of urination. Fluid is drained by gravity or by the patient's use of the abdominal “muscle pump”.

As is conventional with peritoneal dialysis, the infused high osmolarity fluid is typically allowed to dwell in the peritoneal cavity while osmosis occurs to draw fluids, e.g., wastes, from the other portions of the patient and into the peritoneal cavity. The patient may be trained to withhold activating the ascites drain for a period, such as two to five hours, following the scheduled artificial infusion of fluid to the peritoneal cavity. Further, the infusion may automatically occur at night while the patient is asleep.

Devices and methods are disclosed herein for peritoneal transvesicular drainage device fluid removal by reducing the risk of peritonitis, by making barriers to the bacteria and preventing penetration of bacteria into the peritoneal space. The risk of infection can be reduced by infusing fluid through one or more bacterial filters or semipermeable membranes impermeable for bacteria. Such filters, known as Millipore filters, are permeable to the osmotic solute but impermeable to bacteria, and can be incorporated into the design of the infusion fluid path. An example of such filter material can be a filter membrane having a cutoff larger than 20,000 or 60,000 or 100,000 Daltons and impermeable to bacteria (0.2 μm). Osmotic substances such as urea (60 Daltons), creatinine (113 Daltons) and glucose (180 Daltons) can pass through such membrane. Larger pore size filters will allow some human blood compatible proteins such as albumin to pass into the peritoneal space. Proteins can be given as osmotic agent as well as to reduce protein loss caused by the disease and peritoneal fluid removal. Fouling of the membrane, compared to peritoneal dialysis, can be moderated by the smaller amounts of infused fluid and absence of the need to remove body fluid through the filter, since it is removed through the bladder urination route. Various substances that combat bacterial growth can be added to the osmotic infusion fluid. The filter can be placed prior to the port diaphragm or can be between the port body and the tubing carrying the osmotic material from the port to the peritoneum.

The methods disclosed herein may be performed by filling the peritoneal cavity with a physiologically compatible electrolyte solution additionally containing a relatively large molecular weight osmotically effective substance. The electrolyte solution may be infused through the infusion port, an implanted pump with reservoir or infused through a graft (all of which are schematically shown at 603). The electrolyte solution should not be acutely toxic, however substances that are cleared either by the kidney, liver or by other biological mechanism can be used.

Ultrafiltration of excess water in the body to the peritoneal cavity through the body natural membranes is achieved when an osmotic agent is added into the fluid inside the peritoneal space cavity. Osmotic pressure of the cavity fluid is raised over that of plasma fluid, and the removing excess liquid from the patient's body in general becomes possible. For this purpose, solution of glucose has been historically used as an osmotic agent. However, adverse effects such as the dysfunction of the peritoneum due to the absorption of such a large quantity of the osmotic agent into the patient body are now recognized as a serious problem in frequently performed dialysis. With the less frequently performed ultrafiltration such problems can be reduced. It is also recognized that the composition of osmotic fluids for peritoneal dialysis has been extensively researched and many solutions already have been proposed and continue to be experimented with to improve quality of the procedure and protect the patient from complications. Common to all these infusion solutions they have initial osmolarity (the measure of solute concentration) higher than the osmolarity of plasma water, and cause diffusion of water into the peritoneal cavity and dilution of the peritoneal cavity fluid until the two said osmolarites are substantially equilibrated. These infusion solutions may be suitable for infusion through the port 603 to assist in moving body fluids through the valved fistula and into the bladder.

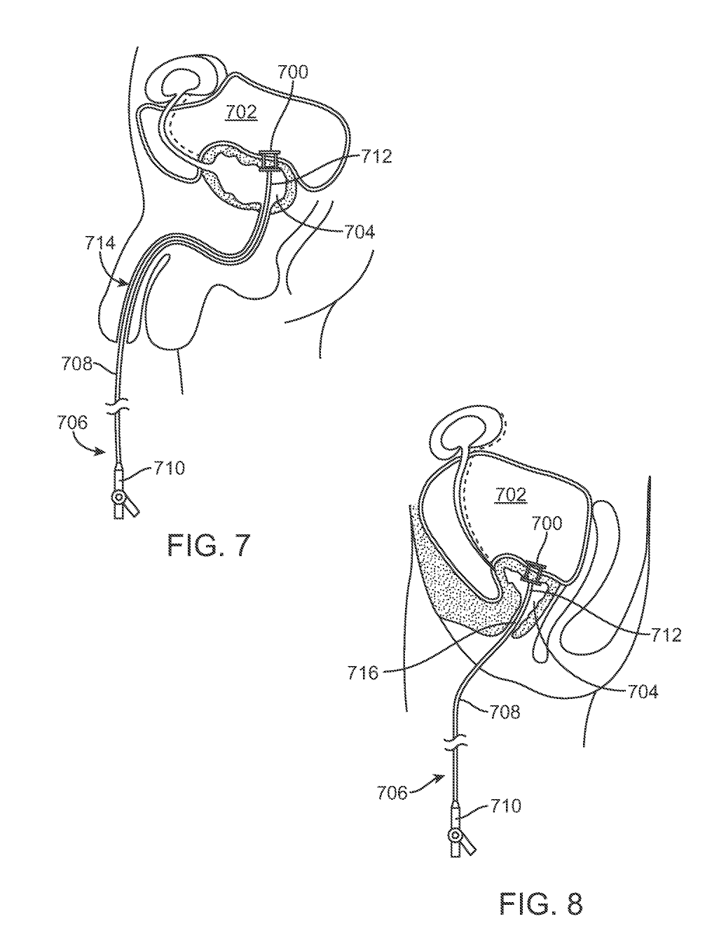

FIGS. 7 and 8 illustrate the insertion and implantation of a valved fistula 700 which drains ascites fluid from the peritoneal cavity 702 to the bladder 704. The insertion and implantation of the fistula 700 is made using a laparoscopic instrument 706 having a multi-lumen catheter 708 and a laparoscopic instrument controller and interface 710. FIG. 7 illustrates a catheter inserted through the urethra 714 of a male patient and FIG. 8 illustrates that catheter 708 inserted through the urethra 716 of a female patient.

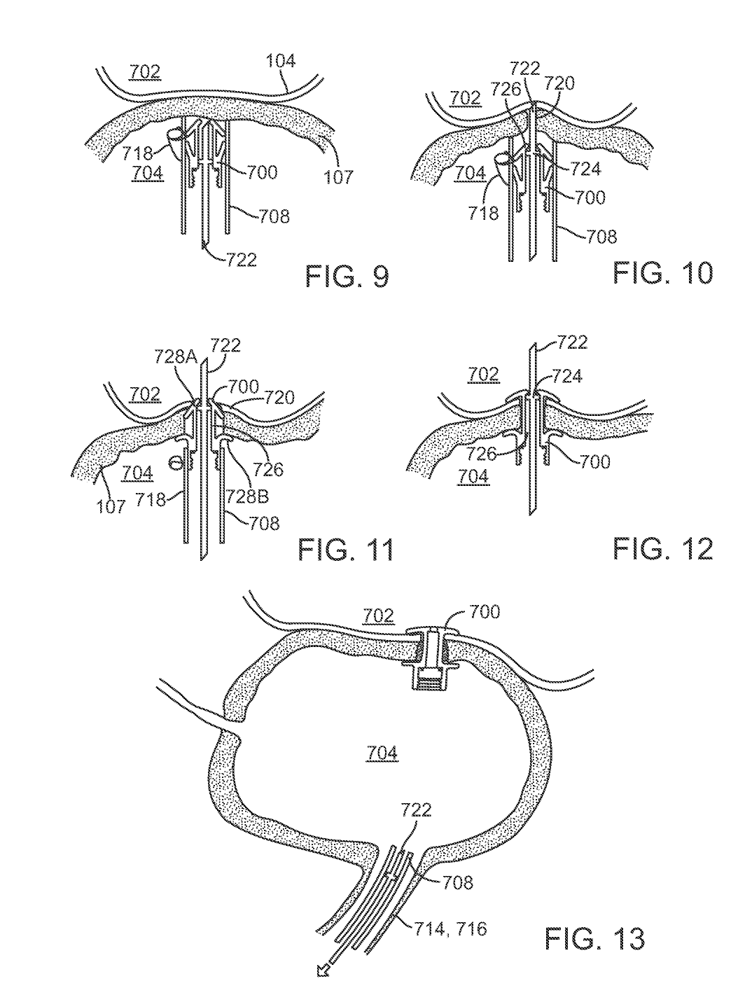

The laparoscopic instrument 706 may include a camera, video or other image capture device 718 (FIG. 9) may be at a distal end section 712 of the catheter and an image presentation assembly, such as a display, video graphics electronics and processing, and a user interface associated with the controller and interface 710. The image capture device may be used to view the muscle wall of the bladder and confirm that the site of the orifice 720 in the muscle wall is suitable for the fistula.

FIGS. 9 to 13 show sequentially the implantation of a valved fistula 700 in the muscle walls 107 of the bladder and the membrane 104 of the peritoneal cavity. As shown in FIG. 9, the distal end of the catheter 708 is moved through the bladder and positioned against the muscle wall 107 where an orifice 720 is to be formed for the fistula. A camera lens 718 may be used to capture an image of the muscle wall. The surgeon may view the captured image to position the distal end of the catheter towards an area of the muscle wall that the surgeon selects for implanting the fistula.

Once the end of the catheter abuts the muscle wall, the valved fistula 700 in a collapsed configuration is advanced through the hollow tube of the catheter 708. A cutting tube or tip 722 may extend through the center passage of the fistula and be used to move the fistula to through the catheter and against the muscle wall.

The cutting tube or tip 722 may have a sharp end which is advanced by the surgeon to puncture the muscle wall and membrane of the peritoneal membrane, as is shown in FIG. 10. The puncture forms the orifice 720 into which the fistula is to be implanted. The cutting tube or tip may by itself form the orifice or the tube of the catheter 708 may be extended into the orifice to expand The orifice before the fistula is implanted (as shown in FIG. 3.

As shown in FIG. 11, the fistula 700 is advanced from the end of the catheter and into the orifice 720. The fistula may be advanced from the catheter by the cutting tube 722. A collar 724 on the shaft of the cutting tube 722 may engage a ledge in the passage 726 of the fistula. The engagement of the collar and ledge allows the cutting tube to push the fistula from the catheter and into the orifice 720.

While in the catheter, the retainers 728a, 728b of the fistula are collapsed so that the fistula can move through the catheter and into the orifice. As the fistula moves from the catheter and into the orifice, the retainers expand. The lower retainer 728b expands radially outward as the retainer moves out of the catheter. The expansion of the lower retainer 728b allows the retainer to quickly seat on the muscle wall 107 and prevent the fistula from being advanced too far into the orifice.

The upper retainer 728a remains collapsed as the fistula moves out of the catheter and through the orifice 720, as is shown in FIG. 11. The upper retainer 728a expands as it moves out of the orifice and passes through the membrane 104 of the peritoneal cavity. Once the upper retainer 728a is seated on the membrane of the peritoneal cavity and the lower retainer 728b is seated on the muscle wall of the bladder, the fistula is secure in the orifice 720, as shown in FIG. 12. The catheter may be retracted from the fistula and remain in the bladder, while the cutting tube 722 or a guidewire remains extending through the fistula. The surgeon may use the camera 718 on the catheter to inspect the installation of the fistula and advance the cutting tube to adjust the positioning of the fistula in the orifice. Thereafter, the cutting tube 722 is retracted into the catheter 708, and the catheter is withdrawn from the bladder 704 and the urethra 714, 716, as is shown in FIG. 13.

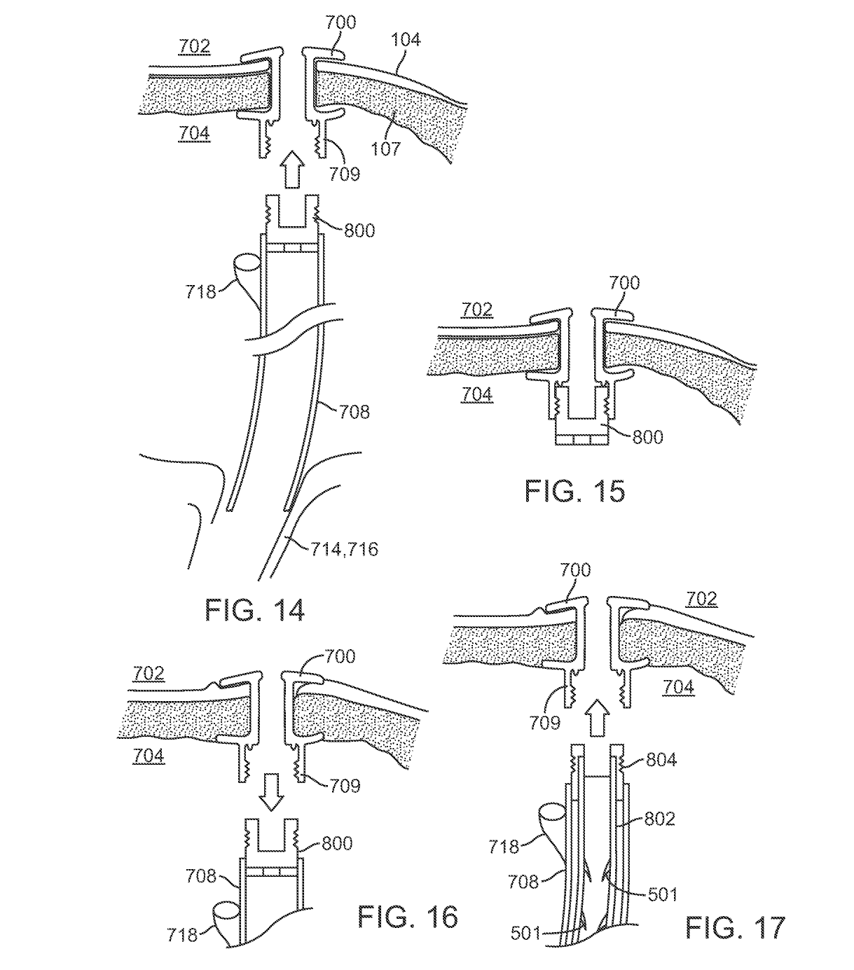

FIGS. 14 to 18 sequentially illustrate the attachment of a plug 800 to temporarily seal the passage 726 in the fistula 700, the removal of the plug and the attachment of the drain tube 802 with internal valves 501. The plug 800 has threaded cylinder that mates with a threaded aperture in the fistula 700. The plug is mounted on the coupling end 709 of the catheter 708 and positioned at the end of the fistula, such as by using images of the fistula captured by the camera 718. The plug is secured to the fistula by rotating the catheter. The plug seals the fistula while muscle wall 107 and membrane 104 of the peritoneal cavity heal around the fistula. The plug may be installed immediately after the fistula is implanted in the body of the patient. The plug is removed after the muscle and membrane heal around the fistula.

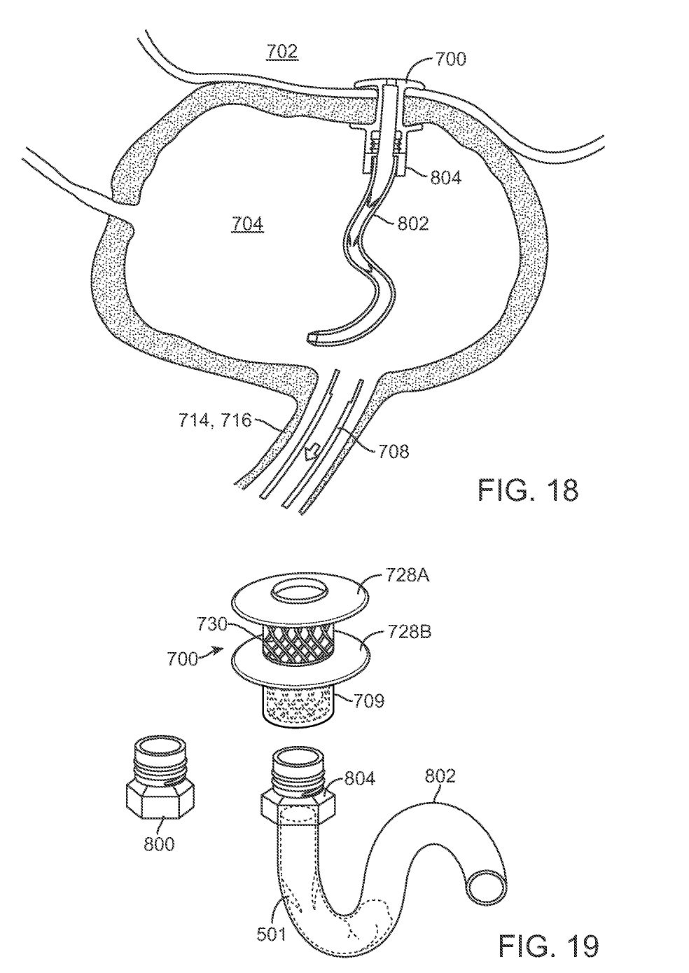

Once the plug is removed, a drainage tube 802 with internal valves 501 is slid into the distal end of the catheter 708. An attachment coupling 804 at a distal end of the tube 802 is connected to the coupling end 709 of the fistula 700. The coupling 804 may have external threads that engage with the internal threads in the coupling end 709 of the fistula. The coupling and tube 802 are rotated by the catheter 708 connect the coupling and tube to the fistula. One the tube is connected to the fistula, the catheter 708 slides off the drain tube and is removed, as shown in FIG. 18.

FIG. 19 shows the fistula 700, plug 800 and drain tube 802. The fistula may include a cylindrical inner structure 730 which may be a mesh of biocompatible material. The mesh may be exposed between the disc retainers 728A, B. The exposed mesh may be coated with materials that promote sealing of the tissue of the muscle and membrane on the surface of the mesh. The portion of the mesh at the attachment coupling may be covered with a biocompatible relatively rigid material which provides structural support for the internal threads and for the plug and attachment coupling 804.

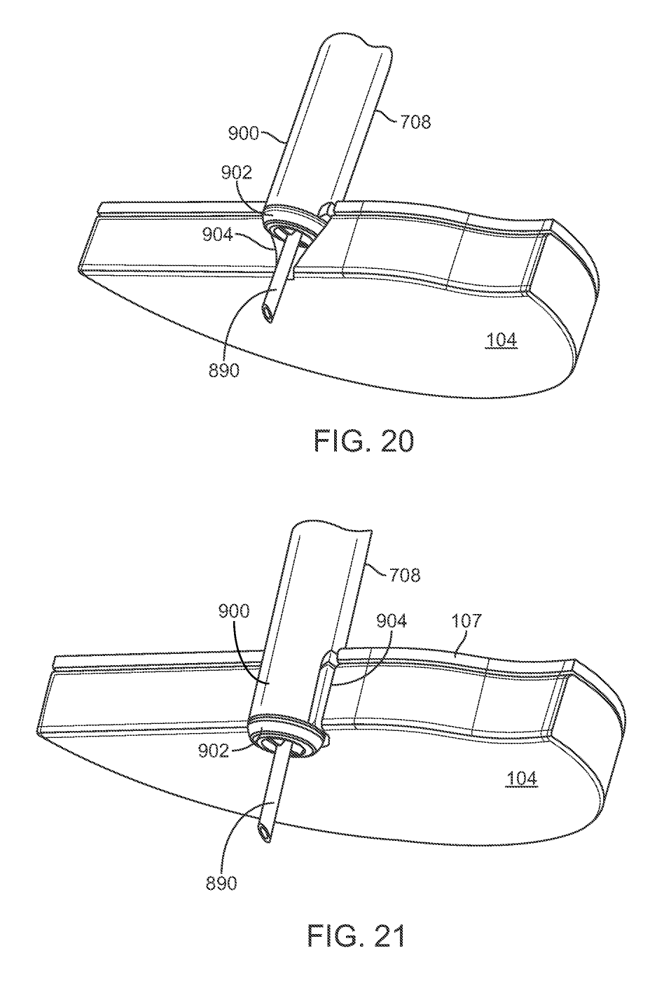

FIGS. 20 to 27 illustrate the implantation of a fistula 895 being implanted into the bladder wall. As shown in FIG. 20, the distal end 900 of the catheter 708 is moved laparoscopically through the abdomen and into the bladder. The distal end 900 of the catheter is positioned in the bladder to abut the bladder wall 900. An annular collar 902 of the fistula is seated on the distal end of the catheter and is pressed against the bladder wall. A hollow cutting tube 890 extends axially through the catheter and fistula. The end tip of the cutting tube may be retracted in the distal end 901 of the catheter until the collar 902 and distal end of the catheter are properly positioned against the bladder wall. The cutting tube 890 is extended to pierce the bladder wall, associated muscles and extend through the peritoneal membrane 104.

As shown in FIGS. 20 and 21, the hole 904 in the bladder wall and associated muscles started by the tip of the cutting tube 890 is expanded as the distal end 900 of the catheter is pushed through the hole. Determining when the distal end 900 pushes through the peritoneal membrane 104 may be achieved by various methods including measuring the distance the catheter is advanced after the end of the catheter has been positioned against the bladder wall, detecting an increase in the resistance to the advance of the catheter as the end of the catheter moves through the muscle wall and the decrease in resistance as the end of the catheter passes the peritoneal membrane, and visually monitoring an image captured by a camera distal end of the catheter. For example, as the surgeon pushes proximal end of the catheter, the surgeon will feel the resistance as the distal end of the catheter pushes through the bladder wall and muscle. The resistance felt by the surgeon noticeably drops off as the distal end protrudes through the peritoneal membrane.

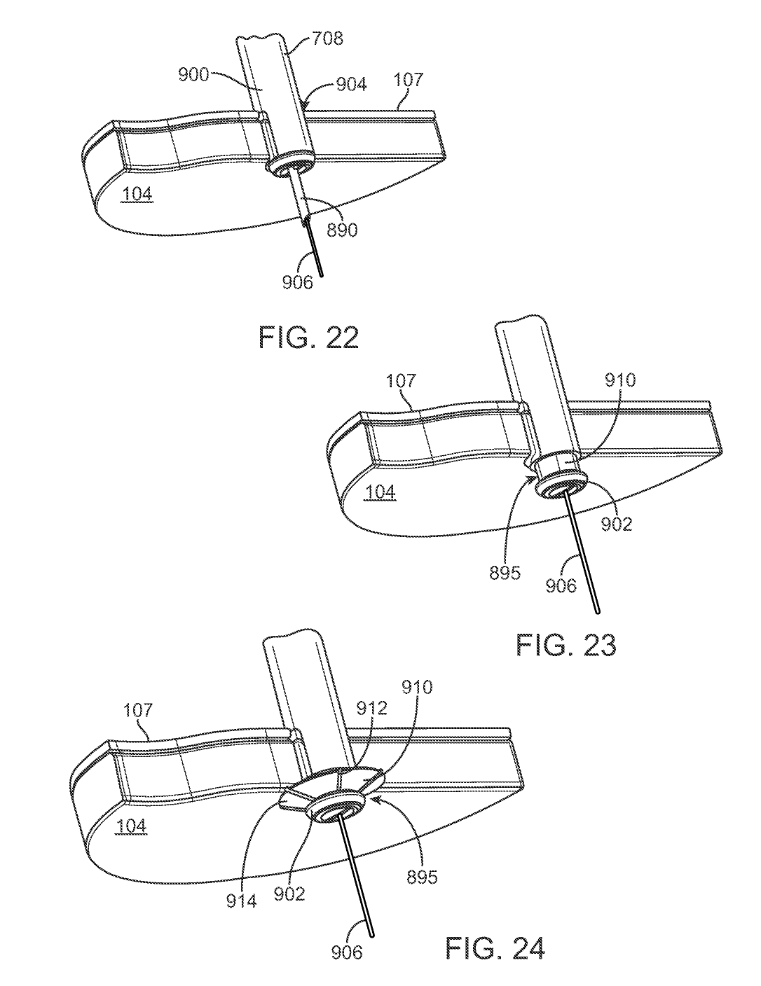

FIG. 22 shows a guide wire 906 extending through the hollow cutting tube 890, and FIG. 23 shows the guide wire remaining after the cutting tube has been retracted into the distal end of the catheter. The fistula may be retained inside the catheter by friction and strength of spring loaded flanges and later pushed out by a “pusher rod” device advanced by the surgeon.

Alternatively, the cutting tube may include an entrapment device that prevents the fistula 895 from being extended from the end of the catheter while the cutting tube is in the center of the fistula.

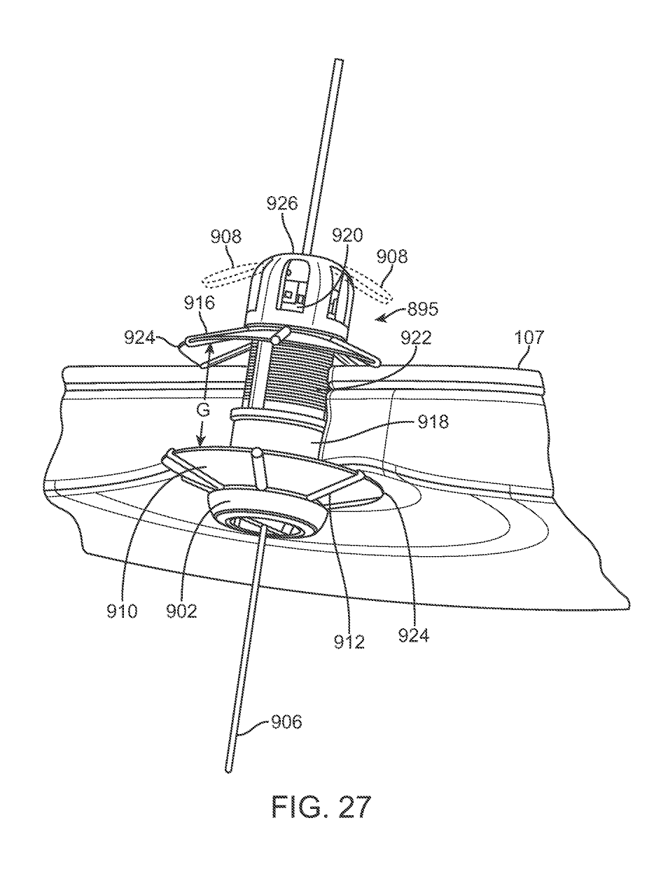

For example, the entrapment device may be a rib, lip or finger 908 (FIG. 27) on the fistula that is extended by the presence of the cutting tube in the fistula. The rib, lip or finger may engage an annular groove on the inside cylindrical surface of the catheter to secure the position of the fistula at the end of the catheter while the cutting tube extends through the fistula. As the cutting tube slides back and out of the fistula, the rib 908, lip or finger retracts within the fistula or within a connector 920 releasably attached to the fistula, as is shown in FIG. 27.

As shown in FIGS. 23 and 24, the fistula 895 may be slid, deployed, pushed out of the distal end of the catheter after the cutting tube is retracted. The fistula includes an annular umbrella securement device 910 which is adjacent the collar 902 and remains closed while the fistula is in the catheter.

The collar 902 may support the ends of elastic ribs 912 on the securement device 910. The ribs extend radially and support a flexible skirt 914 of the securement device. The radially inward ends of the ribs are supported by collar 902 such that ribs spring radially outward from the fistula when the ribs are not confined in the distal end of the catheter.

The collar 902 may also be raised above the skirt on the securement 910 by a gap 915. The annular gap 915 provides an entrance for fluid, e.g., ascites, flowing from the peritoneal cavity into the fistula. The cap prevents solids, such as fat and gut, from entering the fistula and obstructing the fluid passage in the fistula. Alternatively or in addition, the collar may have an axial opening to receive fluid entering the fluid passage in the fistula.

As the fistula slides out of the catheter, the securement device opens as is shown in FIG. 24. The securement device seats on the surface of the peritoneal membrane 104. To seat the opened securement device 901 on the membrane 104, the catheter can be retracted a few millimeters to pull the fistula and securement device towards the membrane. Once seated on the membrane, the securement device anchors the fistula to the membrane.

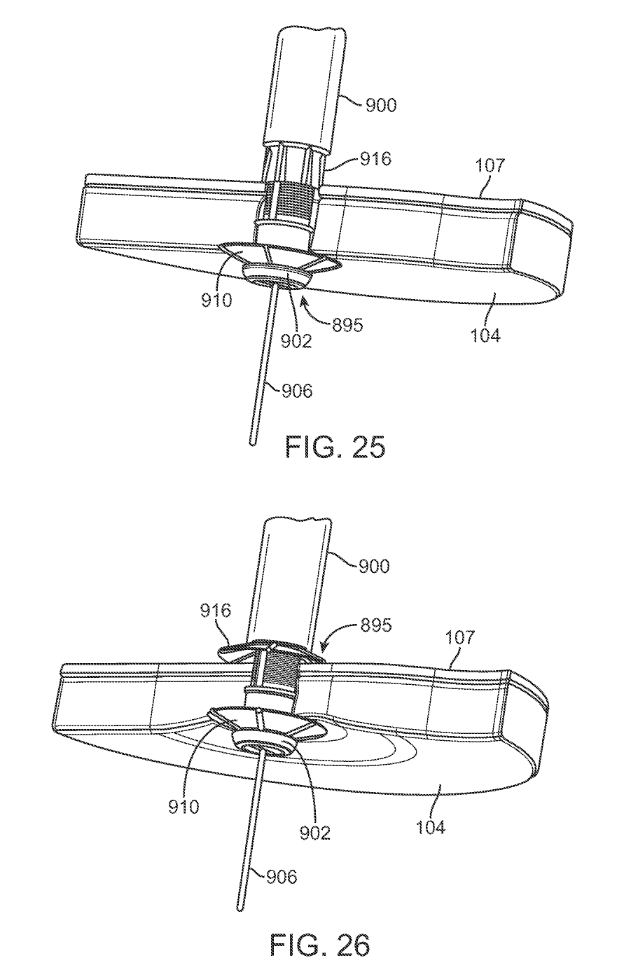

As the retraction of the catheter continues and the securement device 910 is seated on the peritoneal membrane, the remainder of the fistula is withdrawn from the catheter, as is shown in FIG. 25. A second securement device 916 on the fistula opens when released from confinement from inside of the catheter. The second securement device 916 is structurally similar to the first securement device 910 but is oriented to open in an opposite direction from the opening direction of the first securement device. When opened, the second securement device 916 seats against the bladder wall 107, as is shown in FIG. 26.

As is shown in FIG. 27, the fistula 895 includes a cylindrical hollow core 918 having the collar 902 at one end and a connector 920 with a threaded connection at the other end. At least a portion of the outer sidewall of the core 918 may be threaded 922. The collar for the second securement device 916 may have an inner cylindrical surface with threads to engage the threads 922 on the core of the fistula. A Surgeon sets the gap (G) between the securement devices 910, 916 by turning the second securement device about the core and thereby move axially the second securement device on the core. The surgeon sets the gap (G) to be sufficiently wide to allow the perimeter 924 of the second securement device to clear the wall 107 of the bladder as the catheter is retracted. The gap (G) should be sufficiently narrow to seat on the wall 107 of the bladder when the second securement device is opened.

The perimeter 924 of each of the securement devices is a sufficient diameter to engage the surface of the peritoneal membrane or bladder wall and provide an anchor for the fistula and valve tube. The perimeter should be sufficiently small in diameter to allow the securement device to open when released from the distal end of the catheter.

When the fistula 895 is secured between the wall bladder and peritoneal membrane, the end cap 926 may be removed from the end of the core 918 of the fistula. The end cap may be removed by being rotated by the end of the catheter or other tool slid over the guide wire 906. The removal technique shown in FIG. 16 may be employed to remove the end cap. After the end cap is removed by being slide along the guide wire, a valve tube may be attached to the end of the core 918 of the fistula as is illustrated in FIG. 17.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A valved fistula comprising:
a retention element adapted to seat on at least one of a wall a peritoneal cavity and a wall of a bladder of a mammalian patient;
a passage extending between the wall of the peritoneal cavity and the wall of the bladder, and
a valve coupled to the passage, wherein the valve is a one-way valve permitting fluid flow from the peritoneal cavity to the bladder and blocking fluid flow from the bladder to the to the peritoneal cavity, wherein the valve is activated by a pressure differential between the peritoneal cavity and the bladder.

2. The valved fistula of claim 1 wherein the passage includes a tube in the bladder and having an inlet connected to an outlet of the passage.

3. The valved fistula of claim 1 wherein the retention element includes an annular rim extending radially outward from the passage.

4. The valved fistula of claim wherein the retention device includes a deformable

5. A system to drain ascites:
a valve assembly having a retention element adapted to seat on at least one of a wall a peritoneal cavity and a wall of a bladder of a mammalian patient;
the valve assembly further including a passage extending between the wall of the peritoneal cavity and the wall of the bladder;
the valve assembly further including a one-way coupled to the passage, wherein the one-way valve permitting fluid flow from the peritoneal cavity to the bladder and blocking fluid flow from the bladder to the peritoneal cavity, and
an osmotic fluid infusion device adapted to infuse osmotic fluid into the peritoneal cavity to promote the evacuation of ascites from the peritoneal cavity through the valve assembly and through the bladder.

6. A method to remove ascites from a mammalian body comprising:
draining ascites from the peritoneal cavity to the bladder of the mammalian body, and
evacuating the drained ascites out of the bladder.

7. The method of claim 6 wherein the ascites is drained through a one-way valve implanted in the bladder and forming a passage between the peritoneal cavity and bladder, wherein the method includes opening the valve to allow the ascites to drain from the peritoneal cavity.

8. The method of claims 6 further comprising infusing an osmotic fluid into the peritoneal cavity to promote the drainage of the ascites from the peritoneal cavity.

9. A method to drain body fluids from a mammalian patient comprising:
artificially infusing a fluid into the peritoneal cavity;
the artificial infusion of the fluid draws body fluid in the patient to the peritoneal cavity of the patient so as to increase an amount of fluid in the peritoneal cavity;
draining the fluid in the peritoneal cavity through a drain extending between the peritoneal cavity and a bladder of the patient, and
discharging the drained fluid in the bladder by natural urination from the bladder.

10. The method of claim 9 wherein the infused fluid has a higher osmolarity than fluid naturally in the peritoneal cavity.

11. The method of claim 9 wherein the artificial infusion of the fluid in performed by an infusion port having a fluid discharge into the peritoneal cavity.

12. The method of claim 11 wherein the infusion port includes an implanted pump and fluid reservoir, wherein the infused fluid is pumped from the implanted reservoir into the peritoneal cavity.

13. The method of claim 12 wherein the implanted pump is controlled to pump fluid from the reservoir at a predetermined pumping rate or in accordance with a predetermined pumping schedule.

14. The method of claim wherein the method performs a peritoneal dialysis process on the patient.

15. A valved fistula comprising:
a retention element adapted to seat on at least one of a wall a peritoneal cavity and a wall of a bladder of a mammalian patient;
a passage extending between the wall of the peritoneal cavity and the wall of the bladder, and
a valve coupled to the passage, wherein the valve is a one-way valve permitting fluid flow from the peritoneal cavity to the bladder and blocking fluid flow from the bladder to the A valved fistula comprising:
a retention element adapted to seat on at least one of a wall a peritoneal cavity and a wall of a bladder of a mammalian patient;
a passage extending between the wall of the peritoneal cavity and the wall of the bladder, and
a valve coupled to the passage, wherein the valve is a one-way valve permitting fluid flow from the peritoneal cavity to the bladder and blocking fluid flow from the bladder to the peritoneal cavity.

16. The valved fistula of claim 15 wherein the passage includes a tube in the bladder and having an inlet connected to an outlet of the passage.

17. A system to drain ascites:
a valve assembly having a retention element adapted to seat on at least one of a wall a peritoneal cavity and a wall of a bladder of a mammalian patient;
the valve assembly further including a passage extending between the wall of the peritoneal cavity and the wall of the bladder;
the valve assembly further including a one-way coupled to the passage, wherein the one-way valve permitting fluid flow from the peritoneal cavity to the bladder and blocking fluid flow from the bladder to the peritoneal cavity, and
an osmotic fluid infusion device adapted to infuse osmotic fluid into the peritoneal cavity to promote the evacuation of ascites from the peritoneal cavity through the valve assembly and through the bladder.

18. A method to remove ascites from a mammalian body comprising:
draining ascites from the peritoneal cavity to the bladder of the mammalian body, and
evacuating the drained ascites out of the bladder.

19. The method of claim 18 wherein the ascites is drained through a one-way valve implanted in the bladder and forming a passage between the peritoneal cavity and bladder, wherein the method includes opening the valve to allow the ascites to drain from the peritoneal cavity.

20. The method of claims 18 further comprising infusing an osmotic fluid into the peritoneal cavity to promote the drainage of the ascites from the peritoneal cavity.
